(19) United States
(12) Patent Application Publication
(10) Pub. No.: US 2015/0374343 A1
(43) Pub. Date: Dec. 31, 2015

(54) ULTRASOUND IMAGING SYSTEM AND METHOD

(71) Applicant: KONINKLIJKE PHILIPS N.V., Eindhoven (NL)

(72) Inventors: CAIFENG SHAN, EINDHOVEN (NL); FEI ZUO, EINDHOVEN (NL)

(21) Appl. No.: 14/765,356

(22) PCT Filed: Jan. 21, 2014
(86) PCT No.: PCT/IB2014/058419

§ 371 Date: Aug. 3, 2015

Related U.S. Application Data
(60) Provisional application No. 61/763,069, filed on Feb. 11, 2013.

(51) Int. Cl. A61B 8/08; A61B 8/00; A61B 8/14

(52) U.S. Cl. CPC A61B 8/5246; A61B 8/14; A61B 8/429; A61B 8/4254; A61B 8/0858; A61B 8/4483; A61B 8/5223; A61B 8/4444

(57) ABSTRACT

The present invention relates to an ultrasound imaging system (100) comprising: an ultrasound probe (10) that comprises a single element ultrasound transducer (16) for transmitting an receiving ultrasound signals; a movement sensor (18) for sensing a displacement-over-time signal x(t) of a displacement (x) of the ultrasound probe (10) relative to an examination object (24) during signal acquisition; an image acquisition hardware (26) that is configured to reconstruct an M-mode ultrasound image from the received ultrasound signals, said reconstructed M-mode ultrasound image being a two-dimensional image I(t,y) comprising multiple one-dimensional depth signals of substantially constant depth (y) in the examination object (24) illustrated over time (t), wherein the image acquisition hardware (26) is further configured to map said M-mode ultra-sound image I(t,y) to a two-dimensional second image I(x,y) comprising the depth signals illustrated over the displacement (x) by using the displacement-over-time signal x(t) that is sensed with the movement sensor (18); and an image analysis unit (48) that is configured to analyse said second image and to detect at least one tissue layer boundary of the examination object (24) in said second image.

### FIELD OF THE INVENTION

The present invention relates to an ultrasound imaging system. The present invention particularly relates to an ultrasound imaging system for detecting tissue layer boundaries within an examination object. Further, the present invention relates to a method for detecting at least one tissue layer boundary of an examination object. Still further, the present invention relates to a corresponding computer program for implementing the method.

### BACKGROUND OF THE INVENTION

In the field of performance sports, personal fitness and health care appliances it is desirable to get insight into a body's proportional composition of different tissue types. For this purpose it is necessary to distinguish several main tissues from each other. The most important tissues to detect from a health perspective are: fat mass and fat-free mass, lean body mass and muscle mass and a further discrimination of subcutaneous adipose tissue (SAT) and visceral adipose tissue (VAT). The composition and size of these tissue types are good indicators for the physical fitness of a user.

Low levels of physical activity and bad dietary habits can lead to poor physical fitness and in the long run result in lifestyle-related diseases such as diabetes, hypertension, dyslipidemia, polycystic ovary syndrome, reproductive abnormalities, sexual dysfunction, heart disease and metabolic syndrome. Medical professionals have to increasingly deal with these diseases. Having a method for quickly and reliably assessing a patient's level of physical fitness can help the professional to assess to what extent the physical fitness may be impacting the patient's health. Moreover, medically prescribed exercise intervention with fitness level and disease monitoring could be used to improve the patient's health and also document the effectiveness of the treatment. The development of consumer-related health care appliances that may be also used in the home environment would improve the situation, since the patients could then easily examine themselves without the additional help of a doctor.

Many of the commonly used solutions for detecting tissue layers in body tissues use modalities that are too complex to be used in a home setting. Examples are: MRI scan, underwater weighting and skinfold measurements that require proper training to be meaningful. Other state of the art modalities are too inconsistent to provide meaningful data, such as e.g. bioelectrical impedance, which is very sensitive to the varying amount of water in the body. Furthermore, these techniques are only capable of determining total mass of the selected tissue and do not provide insight into thicknesses of certain tissues.

Again other techniques involve measurements with multi-beam or multi-focus ultrasound devices. This however involves heavy processing and costly hardware, which makes those kinds of appliances not useful for the home use.

An ultrasound imaging apparatus for body composition assessment is, for example, disclosed in U.S. Pat. No. 5,941,825. The method disclosed therein proposes to measure body fat by transmitting A-mode ultrasound pulses into the body, measuring at least one reflective distance, selecting the at least one reflective distance, which has the shortest distance, to indicate the distance between the inner and outer border of subcutaneous fat tissue. Selecting the at least one reflective distance corrects for an ultrasound transmission parallax. It is asserted that this allows for a convenient measurement of a layer thickness in the examination object. Tissue layer detection using one-dimensional A-line ultrasound signals has however shown to be relatively imprecise. A-mode ultrasound signals are very sensitive to data noises and less reliable and consistent compared to a two-dimensional ultrasound-based detection.

These problems are according to most prior art devices overcome by using complex transducer probes that include a plurality of transducer elements arranged in a transducer array, which allow to image the inside of the body in a B-mode ultrasound image. Compared to A-mode ultrasound imaging techniques as used in U.S. Pat. No. 5,941,825, these two-dimensional B-mode ultrasound images enable to detect the tissue layers with an increased precision. On the other hand, such complex multi-element transducer arrays are very cost-intensive and therefore do not seem to be meaningful to be used in a home environment for the private use.

### SUMMARY OF THE INVENTION

It is an object of the present invention to provide a device for ultrasound imaging that particularly allows a precise, reliable, fast and cost-effective measurement of tissue layer boundaries within an examination object. Preferably, said device shall be configured to be easily and conveniently operated in a home setting. It is furthermore an object of the present invention to provide a corresponding method for detecting at least one tissue layer boundary of an examination object.

According to a first aspect of the present invention, an ultrasound imaging system is presented that comprises:

an ultrasound probe that comprises a single element ultrasound transducer for transmitting and receiving ultrasound signals;

a movement sensor for sensing a displacement-over-time signal x(t) of a displacement of the ultrasound probe relative to an examination object during signal acquisition;

an image acquisition hardware that is configured to reconstruct an M-mode ultrasound image from the received ultrasound signals, said reconstructed M-mode ultrasound image being a two-dimensional image I(t,y) comprising multiple one-dimensional depth signals of substantially constant depth in the examination object illustrated over time, wherein the image acquisition hardware is further configured to map said M-mode ultrasound image I(t,y) to a two-dimensional second image I(x,y) comprising the depth signals illustrated over the displacement by using the displacement-over-time signal x(t) that is sensed with the movement sensor; and

an image analysis unit that is configured to analyse said second image I(x,y) and to detect at least one tissue layer boundary of the examination object in said second image I(x,y).

According to a second aspect of the present invention, a method for detecting at least one tissue layer boundary of an examination object is presented, wherein said method comprises the steps of:

receiving ultrasound signals of a single element ultrasound transducer;

sensing a displacement-over-time signal x(t) of a displacement of the ultrasound transducer relative to the examination object;

reconstructing an M-mode ultrasound image from the received ultrasound signals, said reconstructed M-mode ultrasound image being a two-dimensional image I(t,y) comprising multiple one-dimensional depth signals of substantially constant depth in the examination object illustrated over time,

mapping said M-mode ultrasound image I(t,y) to a two-dimensional second image I(x,y) comprising the depth signals illustrated over the displacement by using the sensed displacement-over-time signal x(t); and

analysing said second image and detecting at least one tissue layer boundary of the examination object in said second image.

The present invention is based on the idea to provide an ultrasound imaging device that is as cost-effective and as fast as e.g. the skinfold method, but is also highly reliable and consistent in its measurements. This is achieved by using a single element ultrasound transducer that may be integrated in a hand-held device, so that the ultrasound probe may be mechanically (e.g. by hand) moved over a top surface of the examination object. During this movement the included movement sensor senses the displacement of the ultrasound probe relative to the examination object over time. Even though only a single element ultrasound transducer is provided, the presented ultrasound imaging system still allows to reconstruct a two-dimensional image. The presented ultrasound imaging system therefore enables to image a two-dimensional area of the body, so that compared to an on-the-spot measurement, the properties and the thickness of a tissue layer may not only be examined at a distinctive location, but over a whole two-dimensional area of the body. This enables to also examine the spatial development of different tissue layers within the body.

The presented ultrasound imaging system thereto applies an M-mode ultrasound imaging technique, wherein ultrasound pulses are emitted in quick succession over time. During the movement of the ultrasound probe the M-mode image is generated as a composite image of different A-line signals recorded at multiple scan lines with a temporal sampling rate 1/T. This results in a two-dimensional image I(t,y), wherein each of the multiple one-dimensional depth image signal are plotted on the y-axis over the time t on the horizontal axis.

In contrast to “regular” M-mode ultrasound imaging devices, which reconstruct a two-dimensional image showing the depth signal at a distinctive, still-standing point of the body over time, a two-dimensional area scan is reconstructed. This is done as follows: The received two-dimensional depth-over-time M-mode image I(t,y) is mapped to a second depth-over-displacement image I(x,y). This mapping from a two-dimensional I(t,y) image to a two-dimensional I(x,y) image (herein denoted as second image) may be accomplished by taking the displacement-over-time information x(t) into account that is sensed with the integrated movement sensor. In this way the resulting second image shows an image of a two-dimensional area of the examination object similar as in a B-mode image.

In contrast to a B-mode ultrasound image, which is usually generated with a plurality of transducer elements arranged in a transducer array, the presented imaging system allows to produce a comparable two-dimensional image with only one ultrasound transducer element. Using only one ultrasound transducer element of course enables to realize a comparatively cost-efficient overall device. The presented ultrasound imaging system is therefore also suitable for a home setting.

Compared to a very simple A-mode ultrasound imaging device as exemplarily disclosed in U.S. Pat. No. 5,941,825, the presented ultrasound imaging system allows to image the tissue layers and their boundaries over a two-dimensional area instead of only performing an on-the-spot measurement. This significantly increases the reliability of the system and allows to perform very detailed measurements even though only a single element ultrasound transducer is used. Compared to on-the-spot measurements, scanning with the presented ultrasound imaging system allows to measure a volume of body tissue (e.g. fat) under the skin and also enables to e.g. calculate the percentage of fat compared to fat-free tissue.

The at least one tissue layer boundary of the examination object is according to the present invention detected in said second image by applying image analysis techniques, as will be explained further below. This is usually done within the integrated image analysis unit. The image analysis unit may either be hardware or software implemented. By analyzing the second image, the image analysis unit allows to detect at least one tissue layer boundary, preferably a plurality of tissue layer boundaries, so that the thickness of each different tissue layer may be determined by determining the distances between each of the plurality of detected tissue layer boundaries.

Since the presented ultrasound imaging system is used in the M-mode and maps this M-mode ultrasound image to a two-dimensional depth-over-displacement image, several images may be taken at the same displacement position x. M-mode ultrasound images are usually ultrasound videos (frames illustrated over time). If the ultrasound probe is not moved, the produced M-mode image will therefore show a sequence of several depth imaging signals over time which are recorded at one and the same position of the body. Since the presented ultrasound imaging system preferably applies a one-to-one (bijective) mapping, wherein a single depth signal is mapped to a single displacement position, this issue should be overcome.

According to an embodiment of the present invention, the image acquisition hardware is configured to select a processed depth signal for a given displacement position if a plurality of depth signals are received at said displacement position, by averaging said plurality of depth signals or selecting one of the plurality of depth signals that has a highest signal-to-noise ratio, in order to use the selected processed depth signal for mapping said M-mode ultrasound image I(t,y) to the two-dimensional second image I(x,y).

Accordingly, if several depth signals are received on one and the same location of the body, these depth signals are preferably averaged or summed during the mapping. Alternatively, the depth signal with the highest signal-to-noise ratio is selected for the above-described mapping.

According to an embodiment, the ultrasound imaging system further comprises at least one pressure sensor for sensing a pressure with which the ultrasound probe is pressed against a surface of the examination object.

Such a pressure sensor especially has the advantage that differences in the ultrasound image resulting from different applied pressures may be accounted for. The pressure sensor may also be coupled with a visual, audible and/or tactile feedback unit for providing a feedback to the user about the pressure measured with the at least one pressure sensor. In this case, the user may receive an indication if the applied pressure is too high or too low. An audible warning signal may, for example, be generated if the user presses the ultrasound probe against the examination object with a too high pressure that could negatively interfere the measurements. Alternatively, a green light may be provided on the ultrasound probe that turns into a red light if the applied pressure is too high. Such an embodiment is especially advantageous to assist inexperienced users.

In a further preferred embodiment, the ultrasound probe of the ultrasound imaging system comprises a plurality of pressure sensors. This allows to also sense an orientation of the ultrasound probe relative to the examination object. Since the ultrasound imaging system acquires M-mode ultrasound imaging signals and transfers these signals to the above-mentioned second I(x,y) image, it is of utmost importance that the ultrasound probe is arranged substantially perpendicular with respect to the top surface of the examination object. Several pressure sensors that may be spatially distributed over the head of the ultrasound probe may account for this. The pressure sensors may, for example, be arranged at distinctive points of the ultrasound probe which together form an imaginary triangle. If all pressures that are sensed with each of the pressure sensors equal each other, this is an indicator that the ultrasound probe is arranged substantially or exactly perpendicular to the examination object. Through the above-mentioned feedback unit a feedback may also be provided to the user if this is not the case. The user may then correct the orientation of the ultrasound probe relative to the examination object.

For the above-mentioned image mapping it is also of importance that the user preferably moves the transducer probe along a substantially straight line. This may be detected by the above-mentioned movement sensor. According to an embodiment, a plurality of movement sensors, e.g. three movement sensors, may be provided to increase the accuracy of this measurement. This would also allow to sense the displacement of the ultrasound probe in all three spatial dimensions. The above-mentioned feedback unit could also provide a feedback to the user if the ultrasound probe is not correctly moved, i.e. not along a substantially straight line.

In order to detect the tissue layer boundaries within the examination object, the ultrasound imaging system according to the present invention, i.e. the image analysis unit, applies several image analysis and image enhancement techniques. A tissue layer boundary is therein modelled as a connective and/or continuous edge within the ultrasound image.

According to a preferred embodiment, the image analysis unit comprises an edge detector that is configured to detect a plurality of edge points which belong to the at least one tissue layer boundary of the examination object by analyzing a derivative of the depth signal in depth direction in said second image.

This edge detector may be software-implemented. A canny edge detector may e.g. be applied to detect a set of edge points within the second I(x,y) image. Since tissue boundaries usually space horizontally across the ultrasound image, only the derivative in depth-direction (y) is considered in the edge detector. The loose set of edge points obtained using this edge detection may then be merged into groups.

According to an embodiment, the image analysis unit may be configured to compare a length of a detected edge comprising a plurality of detected edge points with a minimum threshold length value. This comparison allows to discard edge points that do most probably not belong to a tissue layer boundary but to other artefacts within the I(x,y) image that are detected by the edge detector. The image analysis unit may be configured to only further process detected edges if their length is above said minimum threshold length value. All others will not be processed further.

In order to avoid false detections due to noises in the raw image data, some image enhancement techniques may be applied.

According to an embodiment of the present invention, the image analysis unit may comprise a filter for filtering said second image using a Gaussian filter. This may smooth the received ultrasound image. The Gaussian smoothing applied in the raw image data could, however, cause the detected edges to shift away from the true tissue layer boundaries. To address this, the accuracy of the edges may be increased by lowering the value of the variance of the Gaussian filter step by step.

According to a preferred embodiment of the present invention, the filter is configured to vary the variance of the Gaussian filter while the edge detector detects the plurality of edge points. This means that at each step of lowering the variance, edge detection is performed by the edge detector and thereby a new set of edge points is produced at a lower variance. The neighborhood of each edge point among the old edge point candidates is now searched whether a neighboring edge point is found that could belong to the same tissue layer boundary. If this is the case, then the old edge point is replaced by the new edge point at the lower variance. In the next step, the image analysis unit may be configured to further decrease the variance of the Gaussian filter and for each detected edge point it is investigated again if there is an adjacent edge point that could belong to the same tissue layer boundary. In this way, the edge points detected by the edge detector are merged together step by step to a continuous edge that indicates the at least one tissue layer boundary within said second I(x,y) image.

According to an embodiment of the present invention, the image analysis unit is configured to merge a number of the detected plurality of edge points, which satisfy a continuity criterion, to at least one continuous edge that at least partly represents the at least one tissue layer boundary. Said continuity criterion may include a length, a depth and a gradient of the at least one continuous edge. This continuity criterion may be modelled as a cost function, based on which a global minimization can be performed to derive the at least one tissue layer boundary based on the detected edge points.

According to an embodiment of the present invention, a kth of the at least one continuous edge C(k) is defined as a set of K₁^{(k)} edge points (xᵢ^{(k)}, yᵢ^{(k)}) which are continuous with respect to a displacement axis (x) in the second image, wherein a length C_{L}(k) of the at least one continuous edge C(k) is defined as C_{L}(k)=K₁^{(k)}, a depth C_{D}(k) of the at least one continuous edge C(k) is defined as

$${C}_{D} ( k ) = \frac{1}{{K}_{1}^{( k )}} {\sum }_{i = 1}^{{K}_{1}^{( k )}} {y}_{i}^{( k )} ,$$

and a gradient C_{G}(k) of the at least one continuous edge (k) is defined as

$${C}_{G} ( k ) = \frac{1}{{K}_{1}^{( k )}} {\sum }_{i = 1}^{{K}_{1}^{( k )}} \vert G ( {x}_{i}^{( k )} , {y}_{i}^{( k )} ) \vert ,$$

and wherein the continuity criterion is defined as: C(k)=w_{L}C_{L}(k)+w_{D}C_{D}(k)+w_{G}C_{G}(k), with w_{L}, w_{D} and w_{G} being weighting factors.

The above-mentioned global minimization allows to model the tissue layer boundaries based on the plurality of edge points that have been detected with the edge detector. The resulting continuous edges that are processed by means of the image analysis unit may sometimes be a segment of the true tissue boundary. Gaps may thus occur between the different detected continuous edges. If no edge points are detected by the edge detector in these gaps, the image analysis unit may be configured to apply an interpolation in order to model the tissue layer boundary in these empty gaps.

According to an embodiment, the image analysis unit is configured to interpolate connection points between different continuous edges if it is detected that said different continuous edges belong to the at least one tissue layer boundary. This interpolation may either be a linear or quadratic interpolation or an interpolation of higher order.

In order to improve the detection of the tissue layer boundaries, the image analysis unit is according to an embodiment of the present invention configured to take body site characteristics into account for improving the detection of the at least one tissue layer boundary.

If the at least one tissue layer boundary is finally detected within the I(x,y) image, the image analysis unit may be configured to calculate a thickness of the at least one tissue layer based on the at least one detected tissue layer boundary.

As already mentioned before, the presented ultrasound imaging system enables to receive a two-dimensional area scan of the examination object. It is thus possible to calculate the thickness of the at least one tissue layer not only at one distinctive spot of the body, but also to calculate the variations of the thickness of the at least one tissue layer throughout the scanned area.

It shall be pointed out again that the present invention does not only relate to the ultrasound imaging system but also to the above-mentioned method for detecting at least one tissue layer boundary of an examination object. It shall be understood that the claimed method has similar and/or identical preferred embodiments as the claimed ultrasound imaging system and as defined in the dependent claims.

According to an embodiment, the claimed method comprises the step of selecting a processed depth signal for a given displacement position if a plurality of depth signals are received at said given displacement position, by averaging said plurality of depth signals or selecting one of the plurality of depth signals that has a highest signal-to-noise ratio, in order to use the selected processed depth signal for mapping said M-mode ultrasound image to the two-dimensional second image.

According to a further embodiment, the claimed method comprises the step of sensing a pressure with which the ultrasound probe is pressed against the surface of the examination object.

According to a further embodiment, the claimed method comprises the step of sensing an orientation of the ultrasound probe relative to a surface of the examination object.

According to a further embodiment, the claimed method comprises the step of detecting a plurality of edge points belonging to the at least one tissue layer boundary of the examination object by analyzing a derivative of the depth signals in depth direction in said image.

According to a further embodiment, the claimed method comprises the step of filtering said second image using a Gaussian filter.

According to a further embodiment, said claimed method comprises the step of varying a variance of the Gaussian filter while the edge detector detects the plurality of edge points.

According to a further embodiment, the claimed method comprises the step of merging a number of the detected plurality of edge points, which satisfy a continuity criterion, to at least one continuous edge that at least partly represents the at least one tissue layer boundary.

According to a further embodiment of the claimed method, said continuity criterion includes a length, a depth and a gradient of the at least one continuous edge. The continuity criterion may be the same as referred to above with respect to the claimed ultrasound imaging system.

According to a further embodiment, the claimed method may comprise the step of interpolating connection points between different continuous edges if it is detected that said different continuous edges belong to the at least one tissue layer boundary.

According to further embodiment, the claimed method comprises the step of calculating a thickness of the at least one tissue layer based on the at least one detected tissue layer boundary.

### BRIEF DESCRIPTION OF THE DRAWINGS

These and other aspects of the invention will be apparent from and elucidated with reference to the embodiment(s) described hereinafter. In the following drawings

FIG. 1 illustrates different views of an ultrasound probe of an ultrasound imaging system according to an embodiment of the present invention;

FIG. 2 schematically illustrates an application of the ultrasound imaging system according to an embodiment of the present invention;

FIG. 3 schematically illustrates a cross section of a human arm;

FIG. 4 shows a schematic block diagram of the ultrasound imaging system according to an embodiment of the present invention;

FIG. 5 shows several ultrasound images received with the ultrasound imaging system in order to illustrate consecutive steps of a tissue layer segmentation performed with the ultrasound imaging system; and

FIG. 6 illustrates an example of a finally processed ultrasound image, in which tissue boundary layers have been detected.

FIG. 7 illustrates a block diagram summarizing the presented method for detecting at least one tissue layer boundary.

### DETAILED DESCRIPTION OF THE INVENTION

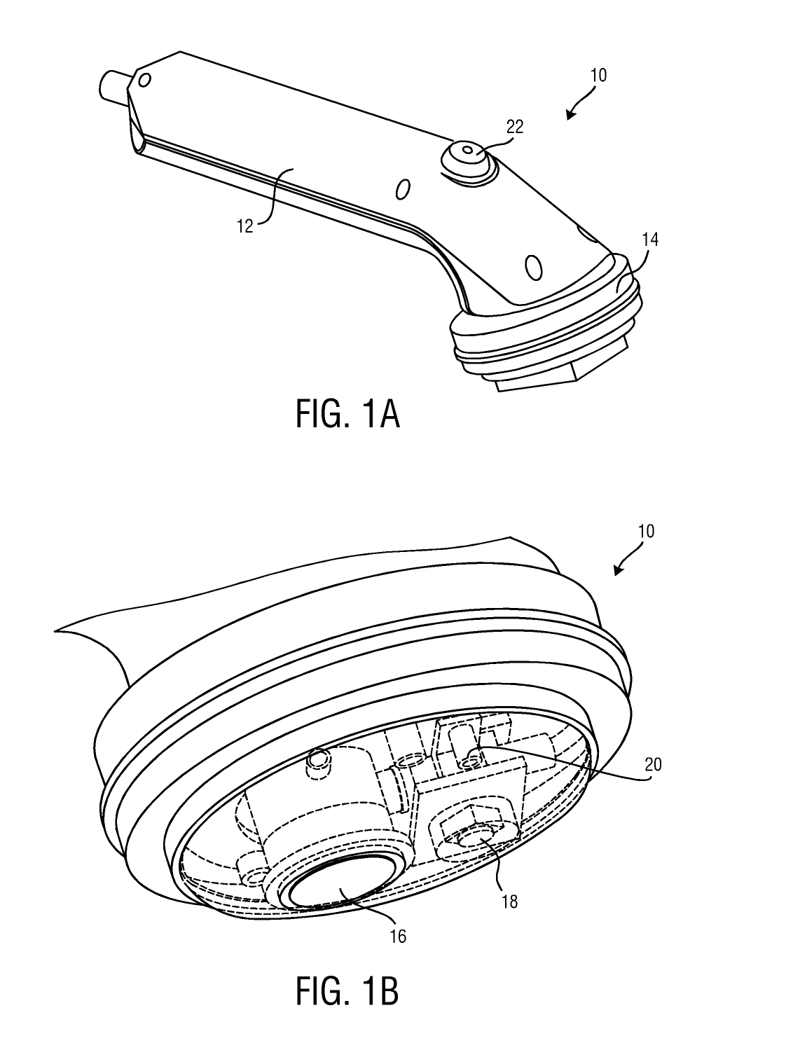

FIG. 1 shows an embodiment of an ultrasound probe 10 of the ultrasound imaging system 100 in two different perspectives. The ultrasound probe 10 is in FIG. 1A shown in its entirety. FIG. 1B shows the head of the ultrasound probe 10 from below. The ultrasound probe 10 comprises a handle 12 and a probe head 14. The probe head 14 in this case has a substantially circular shape. The shape of the probe head 14 may, however, deviate from the illustrated shape without leaving the scope of the invention.

The probe head 14 comprises an ultrasound transducer element 16, a movement sensor 18 and a pressure sensor 20. The ultrasound transducer element 16 is according to the present invention preferably realized as a single element ultrasound transducer 16. This single element ultrasound transducer 16 transmits and receives ultrasound signals. An actuation button 22 may be integrated in the handle 12. This actuation button 22 enables to start and stop the signal acquisition.

The movement sensor 18 is used to detect a displacement of the ultrasound probe 10 relative to an examination object 24 during signal acquisition. This movement sensor 18 is preferably realized as an optical sensor. The optical sensor may, for example, be a similar sensor as the displacement sensors that are used in computer mice. According to an embodiment, the ultrasound probe 10 may feature a plurality of such movement sensors 18. This allows to even more accurately detect the displacement of the ultrasound probe 10 relative to the examination object 24. The movement sensor 18 is preferably configured to detect a displacement of the ultrasound probe 10 relative to the examination object 24 in all three spatial dimensions.

The integrated pressure sensor 20 is configured to sense a pressure with which the ultrasound probe 10 is pressed against the examination object 24. This facilitates to standardize the pressure that is applied between the ultrasound probe 10 and the examination object 24. According to an embodiment, the ultrasound probe 10 comprises a plurality of pressure sensors 20. In case of a provision of at least two pressure sensors 20 this also enables to detect whether the ultrasound probe 10 is arranged correctly (e.g. perpendicularly) relative to the examination object 24.

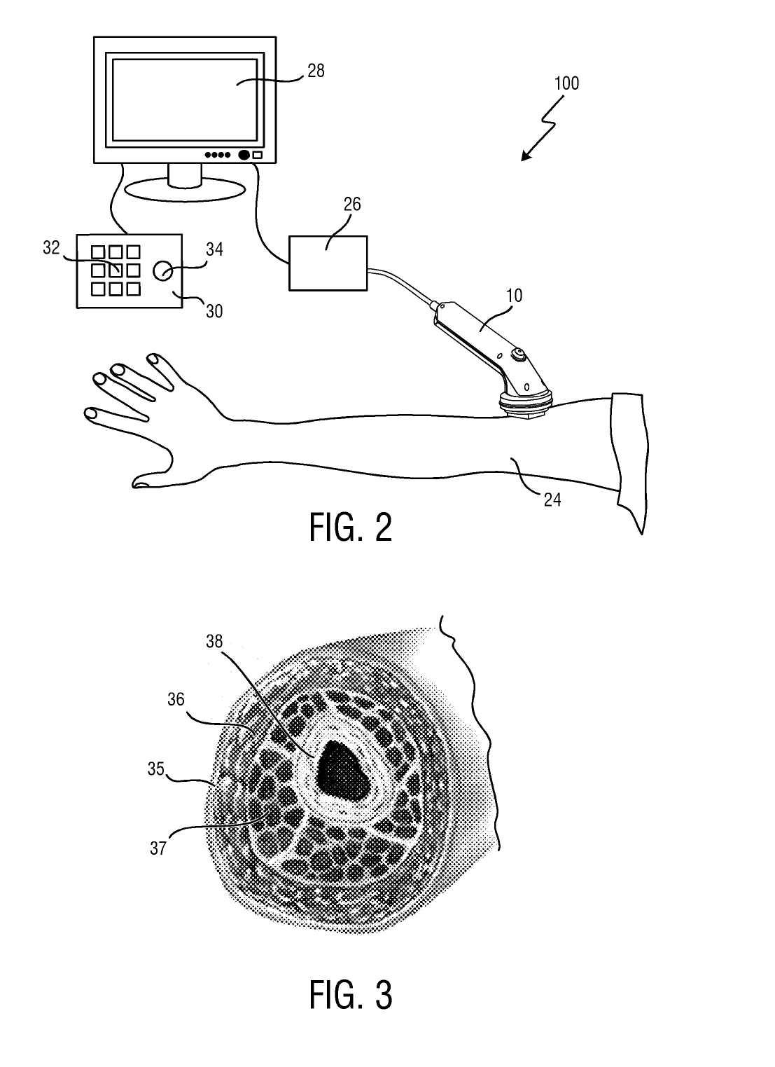

FIG. 2 shows a schematic illustration of the whole ultrasound imaging system 100 according to an embodiment of the present invention. The ultrasound imaging system 100 is applied to inspect a volume of an anatomical site, in particular an anatomical site of an examination object 24 (e.g. a patient 24). The ultrasound imaging system 100 comprises the ultrasound probe 10 that may be hand-held by the user of the system, for example medical staff or a doctor. The presented ultrasound imaging system 100 is designed to be easy in use, such that also private persons may apply the system 100.

The ultrasound imaging system 100 further comprises a controlling unit 26 that controls the provision of an ultrasound image via the ultrasound imaging system 100. As will be explained below in further detail, the controlling unit 26 controls not only the acquisition of data via the ultrasound transducer element 16 of the ultrasound probe 10, but also signal and image processing that form the resulting ultrasound images out of the echoes of the ultrasound beams received by the ultrasound transducer 16.

The ultrasound imaging system 100 further comprises a display 28 for displaying the received ultrasound images to the user. Still further, an input device 30 may be provided that, for example, comprises keys or a keyboard 32 and further inputting devices, for example a trackball 34. The input device 30 may either be connected to the display 28 or directly to the controlling unit 26.

It shall be noted that FIG. 2 is only a schematic illustration. Appliances in practice may deviate from the concrete design shown in FIG. 2 without leaving the scope of the invention. The ultrasound probe 10 and the controlling unit 26 could also be configured as one piece, with our without a display/screen 28, using either a wireless or USB connection to transfer data to a computer for post-processing and calculation purposes. The controlling unit 26 may also be realized as a hand-held device.

The presented ultrasound imaging system is preferably applied for the detection of tissue layers within the examination object 24 by means of ultrasound. As illustrated in FIG. 2, the ultrasound imaging system 100 may, for example, be applied for detecting the different tissue layers within an arm of the patient. FIG. 3 schematically illustrates a cross section through a human arm. The presented ultrasound imaging system 100 may exemplarily be used to image and distinguish between the different tissue layers in the arm, e.g. the skin layer 35, the subcutaneous fat layer 36, the muscle layer 37 and the bone 38.

In order to image and detect the above-mentioned tissue layers, an ultrasound scan is according to the present invention preferably performed by moving the ultrasound probe 10 over a top surface of the examination object 24. During this movement the ultrasound transducer 16 transmits and receives ultrasound signals. As it will be explained further below in detail, an M-mode (motion mode) ultrasound image is thereby generated that is mapped into a two-dimensional area scan image using the displacement information gained with the at least one movement sensor 18. Image analysis and enhancement techniques are then applied in order to detect the different tissue layer boundaries within the processed image. Compared to on-the-spot measurements, this scanning procedure allows to measure the overall volume of body tissue (e.g. fat) under the skin and not only the thickness of said tissue at only one distinctive point.

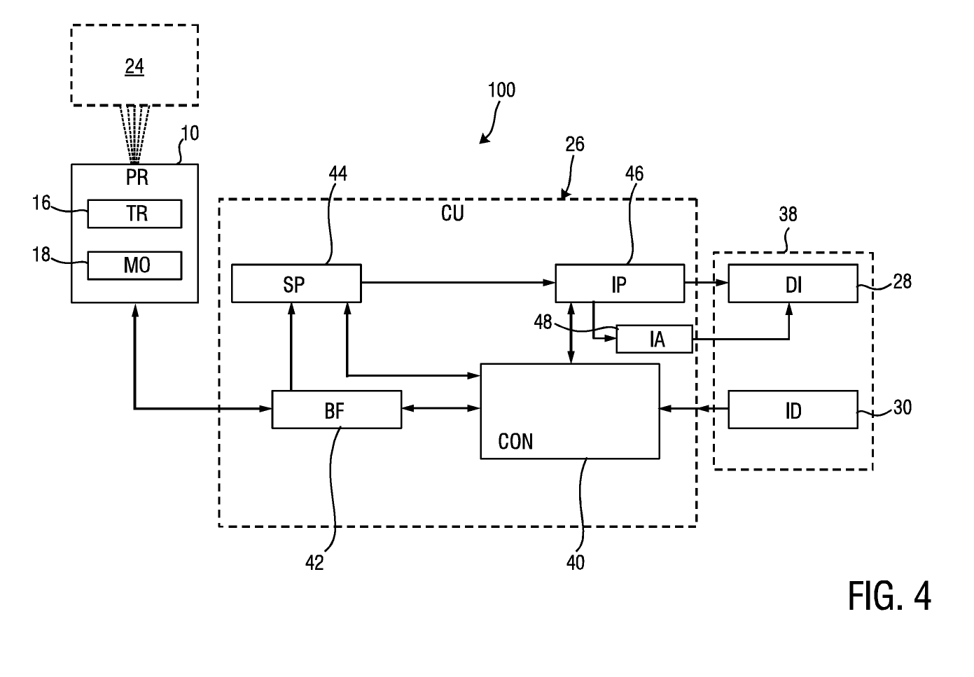

FIG. 4 shows a schematic block diagram of an ultrasound imaging system 100 according to an embodiment of the present invention. It shall be noted that this block diagram is used to illustrate the general concept and design of such an ultrasound system. In practice, the ultrasound imaging system 100 according to the present invention may slightly deviate from the design of this block diagram.

As already laid out above, the ultrasound imaging system 100 comprises the ultrasound probe (PR) 10, the controlling unit (CU) 26, the display (DI) 28 and the input device (ID) 30. The ultrasound probe 10 further comprises the single element ultrasound transducer (TR) 16 for transmitting and receiving ultrasound signals. It further comprises the movement sensor (MO) 18 for sensing the displacement of the ultrasound probe 10 relative to the examination object 24 during signal acquisition. The movement sensor 18 produces a displacement-over-time signal x(t).

In general, the controlling unit 26 may comprise a central processing unit that may include analog and/or digital electronic circuits, a processor, microprocessor or the like to coordinate the whole image acquisition and provision. Further, the controlling unit 26 comprises a herein called image acquisition controller (CON) 40. However, it has to be understood that the image acquisition controller 40 does not need to be a separate entity or unit within the ultrasound imaging system 100. It can be a part of the controlling unit 26 and generally be hardware or software implemented. The current distinction is made for illustrative purposes only. Further, it shall be noted that the controlling unit 26 is herein also referred to as image acquisition hardware 26.

The image acquisition controller 40 as a part of the controlling unit/image acquisition hardware 26 controls a beam former (BF) 42 and by this, what images of the examination object 24 are taken and how these images are taken. The beam former 42 generates voltages that drive the single element ultrasound transducer 16. It may further amplify, filter and digitize the echo voltage stream returned by the transducer element 16.

Further, the image acquisition controller 40 may determine general scanning strategies. Such general strategies may include a desired acquisition rate, lateral extent of the volume, an elevation extent of the volume, maximum and minimum line densities, scanning line times and line density itself. The beam former 42 further receives the ultrasound signals from the transducer element 16 and forwards them as image signals.

Further, the ultrasound imaging system 100 comprises a signal processor (SP) 44 that receives said image signals. The signal processor 44 is generally provided for analog-to-digital converting, digital filtering, for example, bandpass filtering, as well as the detection and compression, for example a dynamic range reduction, of the received ultrasound echoes or image signals. The signal processor 44 forwards image data.

Further, the ultrasound imaging system 100 comprises an image processor (IP) 46 that converts image data received from the signal processor 44 into display data finally shown in the display 28. In particular, the image processor 46 receives the image data, pre-processes the image data and may store it in an image memory (not explicitly shown). These image data are then further post-processed to provide images most convenient to the user via the display 28.

Further, the ultrasound imaging system 100 comprises an image analysis unit (IA) 48 for analzying the reconstructed ultrasound images. Said image analysis unit 48 is either software or hardware implemented and may also be integrated in one of the other components of the controlling unit/image acquisition hardware 26.

In the current case e.g. the image processor 46 forms a M-mode image and transfers this M-mode image into a two-dimensional area scan image I(x,y), which illustrates the depth image signals illustrated over the displacement x of the transducer probe 10. The latter mentioned I(x,y) image is herein also denoted as second image. This transformation shall be shortly explained in the following:

The single element ultrasound transducer 16 is operated in an M-mode. The raw M-mode image that is reconstructed in the image processor 46 of the image acquisition hardware 26 is a composite image of A-line signals that are recorded at multiple scan lines with a temporal sampling rate of 1/T. The M-mode image is a two-dimensional image I(t,y) that comprises multiple one-dimensional depth signals of substantially constant depth y (on the vertical axis) over time t on the horizontal axis. These M-mode ultrasound images may be also referred to as an ultrasound video. In the image processor 46 these M-mode ultrasound images I(t,y) are mapped to a two-dimensional second image I(x,y) that comprises the depth signals y illustrated over the displacement x. With the displacement sensing x(t) from the movement sensor, the time t can be mapped to the displacement x. In case the image processor 46 receives multiple A-line signals at the same displacement position x, the image processor 46 is configured to either average or sum said plurality of A-line signals or to select one of said plurality of A-line signals that has a highest signal to noise ratio. This guarantees a distinct one-to-one mapping. The resulting second image looks similar as a B-mode image that has been taken with a multiple element transducer array, even though according to the present invention only a single transducer element 16 is used. In contrast to a B-mode image the resulting second image does not have the typical cone shape, but a rectangular shape (displacement on the horizontal axis and depth on the vertical axis). This also facilitates the following measurements for detecting the thickness of a tissue layer.

Compared to B-mode images, M-mode images show structures with less detail and have a lower signal-to-noise ratio making the interpretation of these images more difficult. To increase the contrast, the image processor 46 may be configured to apply image enhancement techniques. The image processor 46 may, for example, be configured to map the pixel intensities to new values such that e.g. only 1% of data is saturated at low and high intensities.

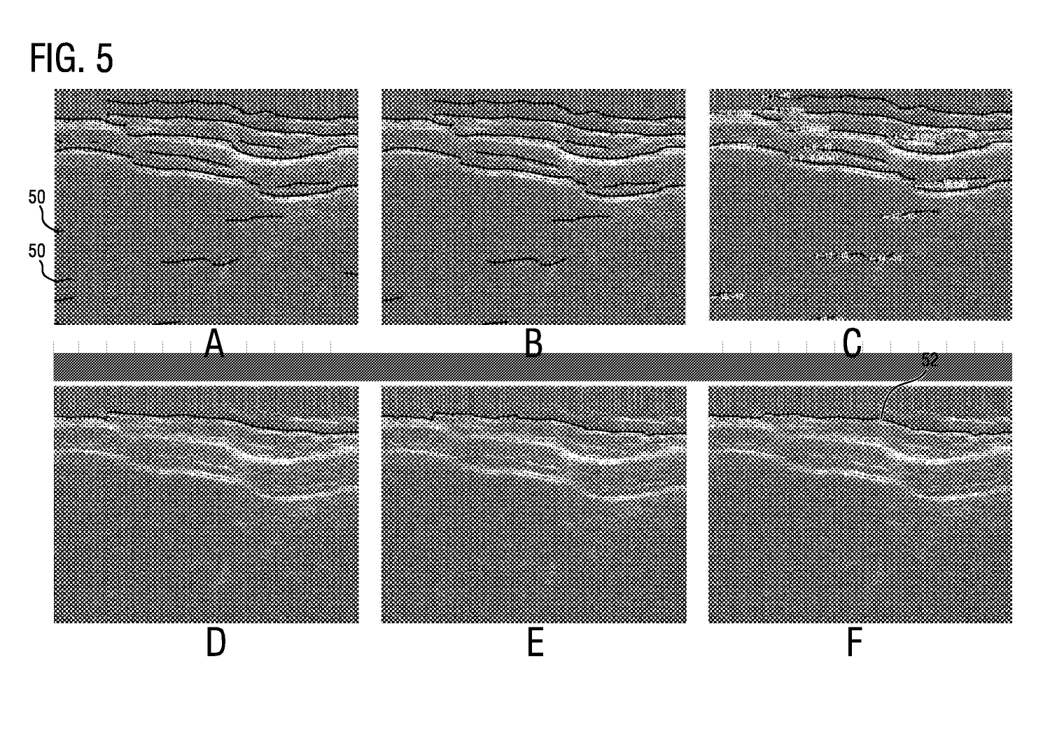

The resulting so-called second I(x,y) images may then be further processed within the image analysis unit 48. This image analysis unit 48 is configured to detect a set of edge points in the ultrasound image (see FIG. 5A). The plurality of edge points may be detected by using an edge detector, such as e.g. a canny edge detector. This edge detector may be configured to analyze a derivative of the depth signal in the depth direction y in said second image I(x,y). To avoid false detections due to noises in the image, the image analysis unit 48 may be configured to smooth the image with a Gaussian filter.

The image analysis unit 48 may be furthermore configured to merge a number of the detected plurality of edge points into groups (see FIG. 5B). Short edges 50 which are below a minimum threshold length may be discarded by the image analysis unit 48 (compare FIGS. 5A and 5B).

In order to model a continuous edge that represents the at least one tissue boundary layer 52, the image analysis unit 48 may be configured to apply a global minimization based on cost function values. This cost function may be herein also denoted as continuity criterion that includes a length, a depth and a gradient of the at least one continuous edge.

Consider a set of K edges. Each edge is a group of merged edge points that have been found by the edge detector (canny edge detection). A kth of the at least one continuous edge C(k) is defined as a set of K ₁^{(k)} edge points (xₜ^{(k)}, yₜ^{(k)}) which are continuous with respect to a displacement axis (x) in the second image, wherein a length C_{L}(k) of the at least one continuous edge C(k) is defined as C_{L}(k)=Kᵢ^{(k)}, a depth C_{D}(k) of the at least one continuous edge C(k) is defined as

$${C}_{D} ( k ) = \frac{1}{{K}_{1}^{( k )}} {\sum }_{i = 1}^{{K}_{1}^{( k )}} {y}_{i}^{( k )} ,$$

and a gradient C_{G}(k) of the at least one continuous edge (k) is defined as

$${C}_{G} ( k ) = \frac{1}{{K}_{1}^{( k )}} {\sum }_{i = 1}^{{K}_{1}^{( k )}} \vert G ( {x}_{i}^{( k )} , {y}_{i}^{( k )} ) \vert ,$$

and wherein the continuity criterion is defined as: C(k)=w_{L}C_{L}(k)+w_{D}C_{d}(k)+w_{G}C_{G}(k), with w_{L}, w_{D} and w_{G} being weighting factors.

The edge chosen by the global minimization is sometimes a segment of the true tissue boundary (see FIG. 5C). The image analysis unit 48 then searches to find other edges satisfying the continuity criterion. If such an edge is found that does not overlap with the chosen edge, the connection of these two edges is merged. The image analysis unit 48 thereto interpolates connection points between the different continuous edges by either a linear or a quadratic interpolation or an interpolation of higher order. This search and interpolation is continued until no more adjacent edges are found that can be connected to the chosen edge. Gaps at both ends of the resulting edge are then continued by keeping respectively the first or the last depth value (see FIG. 5D). Applying the above-mentioned Gaussian filter to the image can cause the detected edges to shift away from the true tissue layer boundary 52. To address this, the image analysis unit 48 may be configured to increase the accuracy of the edges by lowering the value of the variance of the Gaussian filter step by step. At each step edge detection is performed, producing a new set of edge points at a lower variance. The neighborhood of each point among the old edge point candidates is now searched whether an edge point is available. If this is the case, then this point is replaced with the new edge point at the lower variance. In the next step, the variance is further decreased and for each edge point it is again investigated if there is an adjacent edge point in the new set (see FIG. 5E). Finally, an Active Contour Model may be adopted to refine the boundary of the tissue layer (see FIG. 5F). Tissue boundaries can furthermore be enhanced by taking into account the spectrum properties.

The thickness and density of the tissue layers vary between different body sites (and different people). This is due to the fact that tissue matter has varying reflection coefficients, which is caused by factors such as different alignment angles of muscle fiber or tissue depth, resulting in varying visibility of each layers for different body sites. For example, a biceps trajectory is usually characterized by a weak fascia but strong bone boundary, whereas for the calf trajectory a strong inter-muscle boundary can be seen underneath the fascia due to the human anatomy of two layers of calf muscles stacked on top of each other. The above-mentioned tissue layer detection may thus be modified by taking into account the body site characteristics for improved accuracy. The body site information can either be manually selected by the user or automatically detected in the image analysis unit 48.

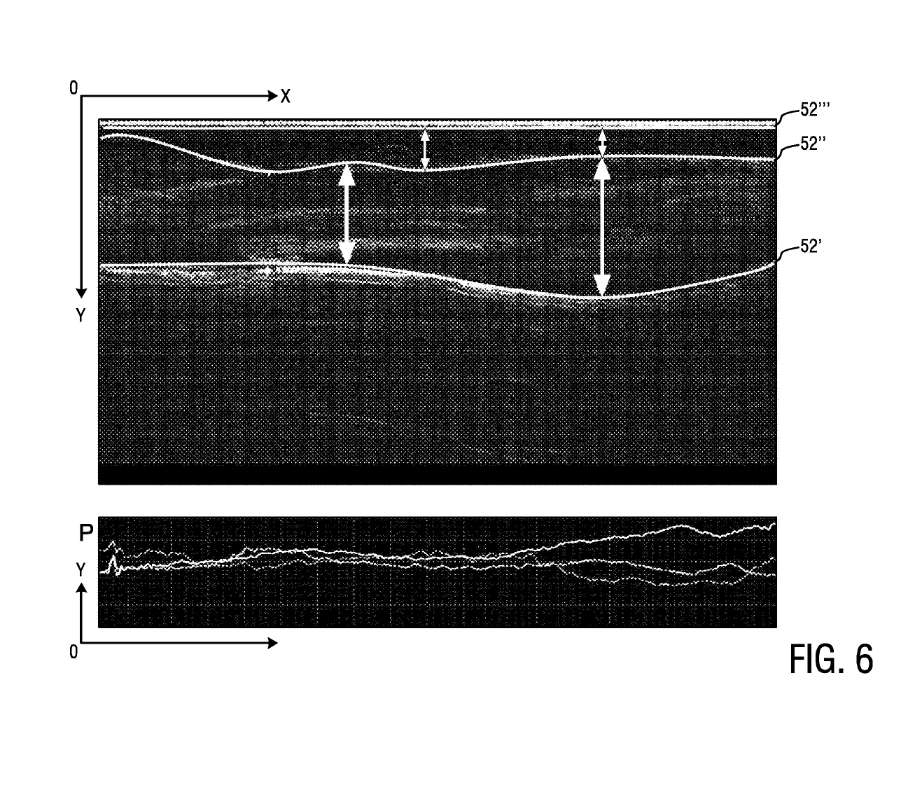

An example of a finally reconstructed ultrasound image with the detected modeled tissue layer boundaries 52 is illustrated in FIG. 6. The upper image illustrated in FIG. 6 shows the I(x,y) image that is herein denoted as second image. The detected layer boundaries therein are the lower boundary 52′ of the muscle layer, the boundary 52″ between the muscle layer and the subcutaneous adipose tissue layer and the boundary 52′″ between the subcutaneous adipose tissue layer and the skin. The illustrated image shows again that it is possible to image the different thicknesses of the tissue layers over the whole scanning region. This is, compared to an on-the-spot measurement in which the thickness of the layers may be only measured on one spot, a significant advantage. Bearing in mind that this image is generated with only a single element ultrasound transducer 16, the present invention enables to accurately determine the thickness layers with a comparatively simple and cheap ultrasound imaging device.

The lower image in FIG. 6 illustrates the pressure that is measured with the above-mentioned pressure sensor 20. In this case three pressure sensors 20 are arranged on different points of the transducer probe head 14. As it can be seen, the pressures measured with these three pressure sensors are, especially in the first part of the image, fairly constant. This is an indicator that the ultrasound probe 10 was arranged almost perpendicular to the top surface of the examination object 24.

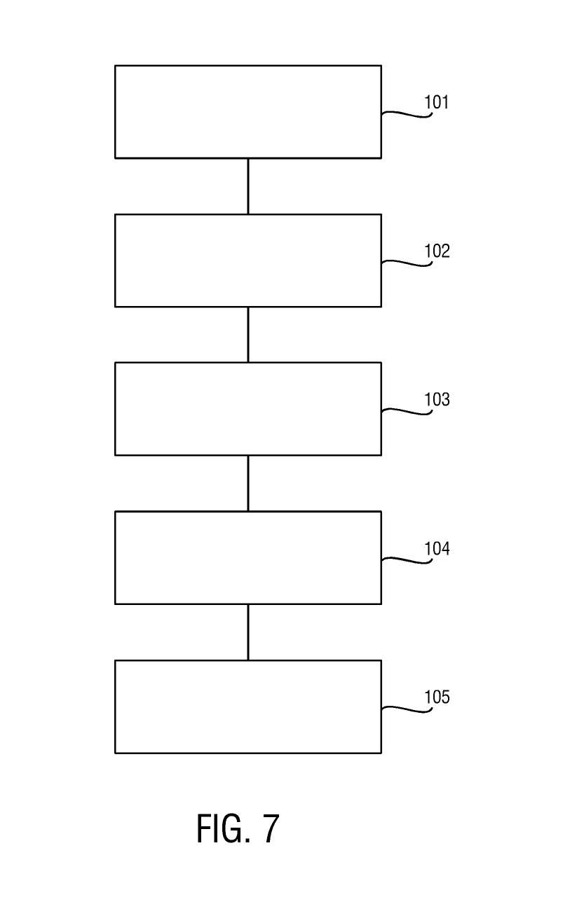

FIG. 7 shows a block diagram summarizing the presented method for detecting at least one tissue layer boundary 52. In a first step 101, ultrasound signals of a single element transducer are received. These ultrasound signals may be either measured in real time or taken from a memory and processed on an external device. In the next step 102, a displacement-over-time signal x(t) of a displacement x of the ultrasound transducer 10 relative to the examination object 24 is sensed. These displacement signals are preferably sensed concurrently with the ultrasound acquisition. Both steps 101, 102 are preferably performed automatically, e.g. by a computer supported ultrasound imaging system. In the third step 103, an M-mode ultrasound image is reconstructed from the received ultrasound signals. Said reconstructed M-mode ultrasound image is a two-dimensional image I(t,y) comprising multiple depth signals of substantially constant depths y in the examination object 24 illustrated over time t. In the following step 104, said M-mode ultrasound image I(t,y) is mapped to a two-dimensional second image I(x,y) comprising the depth signals illustrated over the displacement x by using the sensed displacement over time signal x(t). Finally, said second image I(x,y) is analyzed and at least one tissue layer boundary 52 of the examination object 24 is detected and identified in said second image (step 105).

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. An ultrasound imaging system comprising:
an ultrasound probe that comprises a single element ultrasound transducer for transmitting and receiving ultrasound signals;
a movement sensor for sensing a displacement-over-time signal x(t) of a displacement (x) of the ultrasound probe relative to an examination object during signal acquisition;
an image acquisition hardware that is configured to reconstruct an M-mode ultrasound image from the received ultrasound signals, said reconstructed M-mode ultrasound image being a two-dimensional image I(t,y) comprising multiple one-dimensional depth signals of substantially constant depth (y) in the examination object illustrated over time (t), wherein the image acquisition hardware is further configured to map said M-mode ultrasound image I(t,y) to a two-dimensional second image I(x,y) comprising the depth signals illustrated over the displacement (x) by using the displacement-over-time signal x(t) that is sensed with the movement sensor, wherein if a plurality of depth signals are received at a given displacement position (x), the image acquisition hardware is configured to select a processed depth signal for said given displacement position (x) by averaging said plurality of depth signals or selecting one of the plurality of depth signals that has a highest signal-to-noise ratio, and to use the selected processed depth signal for mapping said M-mode ultrasound image I(t,y) to the two-dimensional second image I(x,y); and
an image analysis unit that is configured to analyse said second image and to detect at least one tissue layer boundary of the examination object in said second image.

2. (canceled)

3. An ultrasound imaging system according to claim 1, further comprising at least one pressure sensor for sensing a pressure with which the ultrasound probe is pressed against a surface of the examination object.

4. An ultrasound imaging system according to claim 1, further comprising multiple pressure sensors for sensing an orientation of the ultrasound probe relative to a surface of the examination object.

5. An ultrasound imaging system according to claim 1, wherein the image analysis unit comprises an edge detector that is configured to detect a plurality of edge points belonging to the at least one tissue layer boundary of the examination object by analysing a derivative of the depth signals in depth direction (y) in said second image.

6. An ultrasound imaging system according to claim 1, wherein the image analysis unit comprises a filter for filtering said second image using a Gaussian filter.

7. An ultrasound imaging system according to claim 6, wherein the filter is configured to vary a variance of the Gaussian filter while the edge detector detects the plurality of edge points.

8. An ultrasound imaging system according to claim 5, wherein the image analysis unit is configured to merge a number of the detected plurality of edge points, which satisfy a continuity criterion, to at least one continuous edge that at least partly represents the at least one tissue layer boundary.

9. An ultrasound imaging system according to claim 8, wherein said continuity criterion includes a length, a depth and a gradient of the at least one continuous edge. kth

10. An ultrasound imaging system according to claim 8, wherein a kth of the at least one continuous edge C(k) is defined as a set of K₁^{(k)} edge points (xᵢ^{(k)}, yᵢ^{(k)} which are continuous with respect to a displacement axis (x) in the second image, wherein a length C_{L}(k)of the at least one continuous edge C(k) is defined as C_{L}(k)=k₁^{(k)}, a depth C_{D}(k) of the at least one continuous edge C(k) is defined as C_{D}(k)=1/K₁^{(k)}Σᵢ₌₁^{K}^{₁}^{^{(k)}}yᵢ^{(k)}, and a gradient of the at least one continuous edge (k) is defined as C_{G}(k)=1/K₁^{(k)}Σᵢ₌₁^{K}^{₁}^{^{(k)}}|G(xᵢ^{(k)}, yᵢ^{(k)})|, and wherein the continuity criterion is defined as: C(k)=w_{L}C_{L}(k)+w_{D}C_{D}(k)+w_{G}C_{G}(k), with w_{L}, w_{D} and w_{G} being weighting factors.

11. An ultrasound imaging system according to claim 8, wherein the image analysis unit is configured to interpolate connection points between different continuous edges if it is detected that said different continuous edges belong to the at least one tissue layer boundary.

12. An ultrasound imaging system according to claim 1, wherein the image analysis unit is configured to take body site characteristics into account for improving the detection of the at least one tissue layer boundary.

13. An ultrasound imaging system according to claim 1, wherein the image analysis unit is configured to calculate a thickness of at least one tissue layer based on the at least one detected tissue layer boundary.

14. A method for detecting at least one tissue layer boundary of an examination object, comprising the steps of:
receiving ultrasound signals of a single element ultrasound transducer;
sensing a displacement-over-time signal x(t) of a displacement (x) of the ultrasound transducer relative to an examination object;
reconstructing an M-mode ultrasound image from the received ultrasound signals, said reconstructed M-mode ultrasound image being a two-dimensional image I(t,y) comprising multiple one-dimensional depth signals of substantially constant depth (y) in the examination object illustrated over time (t),
mapping said M-mode ultrasound image I(t,y) to a two-dimensional second image I(x,y) comprising the depth signals illustrated over the displacement (x) by using the sensed displacement-over-time signal x(t) wherein, if a plurality of depth signals are received at a given displacement position (x), a processed depth signal is selected for said given displacement position (x) by averaging said plurality of depth signals or selecting one of the of depth signals that has a highest signal-to-noise ratio, and the selected processed depth signal is used for mapping said M-mode ultrasound image I(t,y) to the two-dimensional second image I(x,y); and
analysing said second image and detecting at least one tissue layer boundary of the examination object in said second image.

15. Computer program comprising program code means for causing a computer to carry out the steps of the method as claimed in claim 14 when said computer program is carried out on a computer.
